SEQUENCE LISTING (6 sequences)

SEQ ID NO: 1 - DNA, 118 bp, Homo sapiens
tcagttttcctggattatgcctggcaccattaaagaaaatatcatctttggtgtttcctatgatgaatatagatacagaagcgtcatcaaagcatgccaactagaagaggacatctcc

SEQ ID NO: 2 - DNA, 66 bp, Homo sapiens
acctcaaacagacaccatggtgcacctgactcctgwggagaagtctgccgttactgccctgtgggg

SEQ ID NO: 3 - DNA, 51 bp, Homo sapiens
gttcccaataaaagtgactctcagcragcctcaatgctcccagtgctattc

SEQ ID NO: 4 - DNA, 58 bp, Homo sapiens
tctgtaagagcagatccctggacaggcraggaatacaggtattttgtccttgaagtaa

SEQ ID NO: 5 - DNA, 51 bp, Homo sapiens
gaccagctgttcgtgttctatgatsatgagagtcgccgtgtggagccccga

SEQ ID NO: 6 - DNA, 57 bp, Homo sapiens
ccctggggaagagcagagatatacgtrccaggtggagcacccaggcctggatcagcc

(19) United States
(12) Patent Application Publication
(10) Pub. No.: US 2011/0014622 A1
(43) Pub. Date: Jan. 20, 2011

(54) GENETIC REFERENCE MATERIALS

(75) Inventor: JOHN ROSS HAWKINS, POTTERS BAR HERTFORDSHIRE (GB)

Correspondence Address: KNOBBE MARTENS OLSON & BEAR LLP, 2040 MAIN STREET, FOURTEENTH FLOOR, IRVINE, CA, 92614, US

(73) Assignee: NATIONAL BIOLOGICAL STANDARDS BOARD, POTTERS BAR HERTFORDSHIRE (GB)

(21) Appl. No.: 12/890,358

(22) Filed: Sep. 24, 2010

Related U.S. Application Data
(63) Continuation of application No. 10/582,178, filed on Apr. 27, 2007, now abandoned.

(30) Foreign Application Priority Data
Dec. 9, 2003 (GB) 0328451.0

(51) Int. Cl. C12Q 1/68; C12N 5/071; C12N 5/0781

(52) U.S. Cl. 435/6; 435/325; 435/349

(57) ABSTRACT

The invention provides a genetic reference standard with at least one human genetic reference sequence (having a human DNA sequence containing at least one genetic variant whose presence in the DNA of a human subject is indicative of a pathological condition, a predisposition to a pathological condition, or a predisposition to an adverse reaction to external stimuli, or is indicative of a patient's likely response to a therapeutic intervention, i.e. a variant used in pharmacogenomic analysis) cloned into a non-mammalian animal cell line. There are also provided such reference standards where the human DNA is targeted to specific location in the host genome, using homologous recombination. The invention further provides a method of detecting a genetic variant using such reference standards.

### RELATED APPLICATIONS

This application is a continuation application of U.S. application Ser. No. 10/582,178, filed Apr. 27, 2007, which is the U.S. National Phase of International Application No. PCT/GB2004/005033, filed Dec. 1, 2004, designating the U.S. and published in English on Jun. 23, 2005 as WO 2005/056830, which claims the benefit of British Patent Application No. GB0328451.0, filed Dec. 9, 2003, the disclosure of which is hereby expressly incorporated by reference in its entirety.

### BACKGROUND OF THE INVENTION

1. Field of the Invention

The invention relates to methods of producing and maintaining genetic reference materials for use as controls in genetic testing.

2. Description of the Related Art

In clinical genetic diagnostics, it is of utmost importance that test results are accurate. In pre-natal diagnostics for example, false-positive results may lead to the termination of a normal foetus and a false-negative result may lead to the birth of, or failure to diagnose an affected child. In clinical settings, results of genetic tests may form the basis for clinical intervention, and it is essential therefore that the results obtained are soundly based. Increasingly, genetic tests are also used to identify individuals in a population who may have a pre-disposition to disease states. Knowledge of the pre-disposition may lead to effective prophylactic measures, either through clinical intervention or adjustment of lifestyle factors.

To ensure the reliability of such genetic tests, the use of genetic reference materials allows positive or negative controls to be present in each test, thus validating the results. These reference materials consist of DNA, which thus far has been provided by one of three methods:

1. PCR (polymerase chain reaction) product

2. Plasmid-cloned PCR product

3. Human genomic DNA.

Whilst the use of PCR-produced DNA may seem attractive for production of genetic reference material using the sequence of interest, it is associated with a number of disadvantages. The extremely large quantity of DNA produced by such a technique (way in excess of that found in a typical patient sample) poses a significant contamination risk in a typing laboratory. Furthermore, raw PCR-produced DNA is unstable, leading to a lack of precision in its use. Another problem that occurs is that the reference genetic sequence produced by such a technique is produced in isolation, i.e. without the normal background non-target DNA that would be found in a patient-derived sample. Thus, the nature of such standards is considerably different from the test samples with the risk of artefacts in the assay.

Plasmid-cloned PCR product may also be produced by introducing the human genetic reference sequence sequence in a plasmid into an organism such as E. coli. Whilst such a mechanism may be more stable than raw PCR-derived product, there still remains a contamination risk due to the high level of DNA material produced and questions regarding long-term stability. This lack of stability may lead, among other things, to a loss in quality or quantity of the reference DNA.

The third approach, the use of human genomic DNA is not associated with the problems of contamination and instability seen in the first two methods, but has a number of disadvantages of its own. Firstly, any human genomic DNA sample (unless non-specifically amplified), in the form of human cells, will be rapidly consumed in use, and so an ‘immortalised’ cell line is required. This immortalisation process is time consuming and will usually involve the handling of live Epstein-Bar Virus with the associated potential health risks to the operator. The production of an immortalised cell line in this way also requires the use of fresh patient-derived blood, which is often difficult to obtain.

All three of these approaches require, of course, full informed consent from the patient from which the material is derived.

It is an object of the present invention to provide an alternative source of genetic reference material that can be used to standardise genetic testing.

### SUMMARY OF THE INVENTION

In the summary of the invention that follows, the term “human genetic reference sequence” comprises a human DNA sequence containing at least one genetic variant whose presence in the DNA of a human subject is indicative of a pathological condition, a predisposition to a pathological condition, or a predisposition to an adverse reaction to external stimuli. The said genetic variant comprises a change, insertion or deletion of one or more bases with respect to the most common sequence in a human population, and includes single nucleotide polymorphisms (SNP), mutations, base or sequence insertions, base or sequence deletions and a change in tandem repeat length. The reference sequence is characterised in that its total length is at least 35 bases, and does not exceed 30 kilobases. For some applications, the minimum length of the reference sequence may need to be more than 100 bases, to allow detection in an assay in which the reference is to be used. A length of 500 bases will be sufficient for almost all applications, but, within this teaching, the skilled addressee may readily determine an appropriate length by routine experiment, and without further inventive thought.

The invention provides a genetic reference standard comprising at least one human genetic reference sequence cloned into a non-mammalian animal cell line.

Preferably, the animal cell line is an avian cell line; more preferably a chicken (Gallus spp.) cell line and most preferably the chicken DT40 cell line. Most preferably also, the avian cell line is a B-cell line.

According to any aspect of the invention the at least one human genetic reference sequence is cloned into a dispensable region of the cell's genome.

Also according to any aspect of the invention, the at least one human genetic reference sequence is cloned into a non-expressed region of the cell's genome.

Also according to any aspect of the invention, the cloned cell line is diploid with respect to the human genetic reference sequence.

Also according to any aspect of the invention, the at least one human genetic reference sequence is a plurality of human genetic reference sequences.

Also according to any aspect of the invention, the or each human genetic reference sequence is not a functional chromosome.

There is further provided a method of detecting a genetic variant in a sample containing human DNA comprising:

performing a test, responsive to DNA sequence, on said sample;

performing the same test on a reference sample embodying the genetic variant to be detected;

comparing the test results obtained from said sample and said reference sample to determine the presence or absence of said genetic variant; characterised in that said reference sample is a genetic reference standard as described in any aspect of the invention above.

The invention will be described by reference to the accompanying drawings in which:

### BRIEF DESCRIPTION OF THE DRAWINGS

FIG. 1 illustrates a targeting vector suitable for introducing a human genetic reference sequence into a suitable cell line.

FIG. 2 illustrates a targeting vector suitable for introducing a human genetic reference sequence into a suitable cell line, together with the range of cells so produced; and

FIG. 3 illustrates a targeting vector and a scheme by which heterozygous cell lines may be produced.

### DETAILED DESCRIPTION OF THE PREFERRED EMBODIMENT

Cloning DNA fragments into a heterologous eukaryotic cell line in this way acts as an intermediate form of genetic reference material. Being genomic-based, the material would be both stable, and not present a contamination risk. Furthermore, by not containing human sequences, the background DNA would be less likely to cross-react in any human testing protocol. Patient blood would not be required and the handling of a pathogenic human virus would also be avoided.

The required human genetic reference sequence may be derived from a patient possessing the genotype associated with the test. Alternatively, the sequence may be obtained from any unaffected individual and artificially modified such that the DNA sequence matches that of the mutant or rare form. Thus, the PCR product being cloned may derive from a buccal swab and need not even be patient-derived. Also, the sequence may be derived from a normal human cell line, and engineered to introduce the required variant(s). The use of a cell line derived from an anonymous donor in this way would obviate the requirement for informed consent from a known donor. Furthermore, if the required human genetic reference sequence were of a sufficiently short length, then it could also be synthesised from knowledge of its sequence.

Thus, human genetic reference material may be produced by the cloning of one or more DNA reference sequences into a (heterologous) non-mammalian cell line. The use of homologous recombination methods allows the creation of cell lines with a controlled number of copies of defined human DNA sequences. The use of homologous recombination methods also allows the human DNA sequences to be targeted to a specific location within the host genome.

In order to illustrate typical applications for the invention, we consider some of the many genetic screens for which the invention can provide reference materials. Genetic screening may be carried out for a number of ends, such as:

1. Screening for mutations that cause rare diseases;

2. Screening for DNA variants which predispose to common diseases; and

3. Screening for DNA variants which effect drug response.

Examples of each of these three types are given below. In the partial reference sequences quoted, the altered bases are indicated by the use of lower case letters.

I—Gene Mutations or Variants that Cause Genetic Disorders

### I(i) Cystic Fibrosis

Cystic fibrosis is a common (recessive) monogenic disease in European populations, occurring 1 in around 2500 live births. There are many causative mutations, but in the UK population 9 mutations account for around 83% of the CF mutations [DF508—75.3%; G551D—3.08%; G542X—1.68%; 621+1(G>T)—0.93%; 1717−1(G>A)—0.57%; 1898+1)(G>A)—0.46%; R117H—0.46%; N1303K 0.46%; R553X—0.46%]

### DF508 DNA Sequence (TTT Deletion):

(SEQ ID NO: 1)
TCAGTTTTCCTGGATTATGCCTGGCACCATTAAAGAAAATATCATCtttG
GTGTTTCCTATGATGAATATAGATACAGAAGCGTCATCAAAGCATGCCAA
CTAGAAGAGGACATCTCC.

### I(ii) Sickle Cell Anaemia

Sickle cell anaemia is an inherited blood disorder characterized primarily by chronic anaemia and periodic episodes of pain. Sickle cell anaemia is an autosomal recessive genetic disorder caused by a defect in the beta-haemoglobin gene (HBB). The disease occurs in about 1 in every 500 African-American births and 1 in every 1000 to 1400 Hispanic-American births. Although several hundred HBB gene variants are known, sickle cell anaemia is most commonly caused by the hemoglobin variant Hb S. In this variant (E6V) the amino acid valine takes the place of glutamic acid at the sixth amino acid position of the HBB polypeptide chain.

NCBI SNP Cluster ID: rs334

(SEQ ID NO: 2)
ACCTCAAACAGACACCATGGTGCACCTGACTCCTGa/tGGAGAAGTCTGC
CGTTACTGCCCTGTGGGG.

### I(iii) Myotonic Dystrophy

Myotonic dystrophy is a dominantly inherited disease in which the muscles contract but have decreasing power to relax. With this condition, the muscles also become weak and waste away. Unaffected individuals have between 5 and 27 copies of a ‘CTG triplet repeat’ in the 3′ untranslated region of a protein kinase gene. Myotonic dystrophy patients who are minimally affected have at least 50 repeats, while more severely affected patients have an expansion of up to several kilobase pairs.

II Gene Variants that Predispose to Disorders

### II(i) Factor 2 (Prothrombin)

This is a G-to-A transition variation at position 20210 in the 3′ untranslated region of the prothrombin gene that is associated with elevated plasma prothrombin levels and an increased risk of venous thrombosis. The minor (A) allele is present at a frequency of around 1%, so that individuals heterozygous for the variant occur at a frequency of around 2%. Individuals homozygous for the variant occur very rarely at a frequency of around 1 in 10,000.

NCBI SNP Cluster ID: rs1799963

| | |
|---|---|
| GTTCCCAATAAAAGTGACTCTCAGCg/ | SEQ ID NO: 3) |
| aAGCCTCAATGCTCCCAGTGCTATTC. | |

### II(ii) Factor 5

This is a G-to-A variant at position ‘1691’ causing an Arginine to Glutamine amino acid substitution. Again, this is associated with risk of venous thrombosis. Individuals heterozygous for the variant occur at a frequency of around 5%, and individuals homozygous for the variant occur at a frequency of around 1 in 1650.

NCBI SNP Cluster ID: rs6025

(SEQ ID NO: 4)
TCTGTAAGAGCAGATCCCTGGACAGGCg/aAGGAATACAGGTATTTTGTC
CTTGAAGTAA.

### II(iii) Hereditary Haemochromatosis

Hereditary haemochromatosis is a common (recessive) iron-overload disorder. There are two common mutations: C282Y and H63D. The C282Y mutation results from a G-to-A transition at nucleotide 845 of the HFE gene (845G to A) that produces a substitution of cysteine for a tyrosine at amino acid position 282 in the protein product. In the H63D mutation, a G replaces C at nucleotide 187 of the gene (187C to G), causing aspartate to substitute for histidine at amino acid position 63 in the HFE protein. Individuals homozygous for either of these variants or compound heterozygous have an increased risk of iron overload disease. In the UK population, C282Y has an allele frequency of around 0.07 and H63D has an allele frequency of around 0.14.

### H63D DNA Sequence:

| | |
|---|---|
| GACCAGCTGTTCGTGTTCTATGATc/ | (SEQ ID NO: 5) |
| gATGAGAGTCGCCGTGTGGAGCCCCGA. | |

### C282Y DNA Sequence:

(SEQ ID NO: 6)
CCCTGGGGAAGAGCAGAGATATACGTg/aCCAGGTGGAGCACCCAGGCCT
GGATCAGCC.

### III—Gene Variants Affecting Drug Response

### III(i) Thiopurine S-Methlyltransferase (TPMT)

TPMT gene variation affects an individual's ability to metabolize the thiopurine class of drugs. Studies have shown that one in 300 individuals (0.3%) have low to absent levels of TPMT enzyme activity (homozygous recessive), 11% have intermediate levels of enzyme activity (heterozygous) and 89% have normal to high levels of enzyme activity (homozygous normal). TPMT testing allows physicians to identify, prior to initiating therapy, patients who are at risk for developing acute toxicity to the thiopurine class of drugs.

Four variant TPMT alleles have been identified (TPMT*2, TPMT*3A, TPMT*3B, TPMT*3C), which account for 80% of Caucasians with low or intermediate TPMT activity. TPMT*2 contains a G->C substitution at nucleotide 238, while TPMT*3A contains two nucleotide transition mutations (G460A and A719G). TPMT*3B has only G460A, while TPMT*3C contains only A719G. The Caucasian allele frequencies are: TPMT*2—0.5%; *3A—4.5%; *3C—0.3%

This range of examples of genetic screens will allow the skilled addressee to identify other potential applications for the current inventions. Whilst only short sequences have been identified in the above examples, longer sequences containing the same genetic variants may readily be constructed by reference to the published sequence data, should these be required in any assay using the standard. The genetic variant (e.g. SNP, mutation, deletion, insertion etc.) may conveniently be located in any position in the human genetic reference sequence, as required for any subsequent assay. However, for some assays, it is particularly advantageous to locate the variant towards the centre of the human genetic reference sequence. Preferably also, the human genetic reference sequence is not a functional chromosome (i.e. unable to stably replicate independent of the host genome) and most preferably non-centromeric.

Preferably, the human genetic reference sequence may be cloned into a non-mammalian eukaryotic cell to provide a genomic DNA background that would not be likely to cross react with the genetic test. Potential species include fish, frog, insect, birds and some species of plant. Within fish, zebrafish (Danio rerio) cell lines are particularly suitable, as a large armoury of genetic techniques available for this species. Within the plant kingdom, the moss Physcomitrella patens is a particularly attractive target as it has a naturally high homologous recombination efficiency (see: Bernd R., “Homologous recombination and gene targeting in plant cells”. Int Rev Cytol. 228:85-139, 2003 and Hohe A, Egener T, Lucht J M, Holtorf H, Reinhard C, Schween G & Reski R. “Δn improved and highly standardised transformation procedure allows efficient production of single and multiple targeted gene-knockouts in a moss, Physcomitrella patens.” Curr Genet. 44:339-47, 2004)

Approaches to increase the frequency of recombination could be incorporated, and will be evident to the skilled addressee: examples would include the use of the Cre/loxP system (see e.g. Koike H, Horie K, Fukuyama H, Kondoh G, Nagata S & Takeda J. “Efficient biallelic mutagenesis with Cre/loxP-mediated inter-chromosomal recombination”, EMBO Rep. 3:433-7, 2002; and Bode J, Schlake T, Iber M, Schubeler D, Seibler J, Snezhkov E & Nikolaev L. “The transgeneticist's toolbox: novel methods for the targeted modification of eukaryotic genomes” Biol. Chem. 381:801-13, 2000) and compounds such as PARP inhibitors (Semionov A, Cournoyer D & Chow T Y. “1,5-isoquinolinediol increases the frequency of gene targeting by homologous recombination in mouse fibroblasts”, Biochem Cell Biol. 81:17-24, 2003).

Avian cell lines are also particularly suitable for this purpose, as the genomic DNA is substantially different to that of humans. Of these lines, cells from chicken (Gallus spp.) are an especially advantageous heterologous host, as not only is chicken genomic DNA substantially different to that of humans, but also has a similar size (ca. 1.2 Gigabases for chicken compared with 3.2 Gigabases for humans).

The chicken B-cell line DT40 (Baba et al, Virology 144:139-151, 1985) is a particularly effective cell line for this purpose, as it is highly recombination-efficient and avoids the likelihood of multiple integrant copies and instability associated with random integration. There is also a considerable existing literature on techniques for genetic manipulation of DT40. Using the cells recombination machinery a single DNA molecule may be integrated into a defined position by the use, e.g. of targeting arms. Such techniques will be illustrated in the embodiment below.

In order to mimic the situation that may be encountered in human patient-derived samples, both homozygotes and heterozygotes may be produced as desired.

In order to facilitate the construction of a number of manipulated cell lines for different genetic tests, a single targeting construct, given the teaching of this disclosure, may be constructed that would serve for all required DNA fragments. Alternatively, a pair of constructs with identical targeting arms but different antibiotic resistance genes (see below) may be used for the production of heterozygotes. Finally, by using multiple recombination sites, single cell lines may be produced carrying multiple reference fragments. This approach may be facilitated by the use of mutated LoxP sites, to allow the re-use of antibiotic resistance markers.

Methods for the design of targeting vectors are known to the skilled addressee, and the following sources are identified for reference:
Vasquez K M, Marburger K, Intody Z & Wilson J H. (2001) Manipulating the mammalian genome by homologous recombination. Proc Natl Acad Sci USA. 98:8403-10.
Muller U. (1999) Ten years of gene targeting: targeted mouse mutants, from vector design to phenotype analysis. Mech Dev. 82:3-21.
Dickinson P, Kimber W L, Kilanowski F M, Stevenson B J, Porteous D J & Dorin J R. (1993) High frequency gene targeting using insertional vectors. Hum Mol Genet. 2:1299-302.
Morrow B, Kucherlapati R. (1993) Gene targeting in mammalian cells by homologous recombination. Curr Opin Biotechnol. 4:577-82.
Willnow T E & Herz J. (1994) Homologous recombination for gene replacement in mouse cell lines. Methods Cell Biol. 1994; 43 Pt A:305-34.
Bronson S K, Plaehn E G, Kluckman K D, Hagaman J R, Maeda N & Smithies O. (1996) Single-copy transgenic mice with chosen-site integration. Proc Natl Acad Sci USA. 93:9067-72.
Jacenko O. (1997) Strategies in generating transgenic mammals. Methods Mol Biol. 62:399-424.

Embodiments of the invention will now be described. In these examples, vector construction is by use of restriction endonuclease-based in vitro techniques, but it is envisaged that deviations from the scheme described could include construction of the vectors and the insertion of human sequences into the vectors using E. coli or yeast homologous recombination systems.

### Embodiment 1

This embodiment illustrates a way in which the invention may be worked to create a genetic reference standard by insertion of a human genetic reference sequence into a dispensable region of the genome of the chicken DT40 cell line.

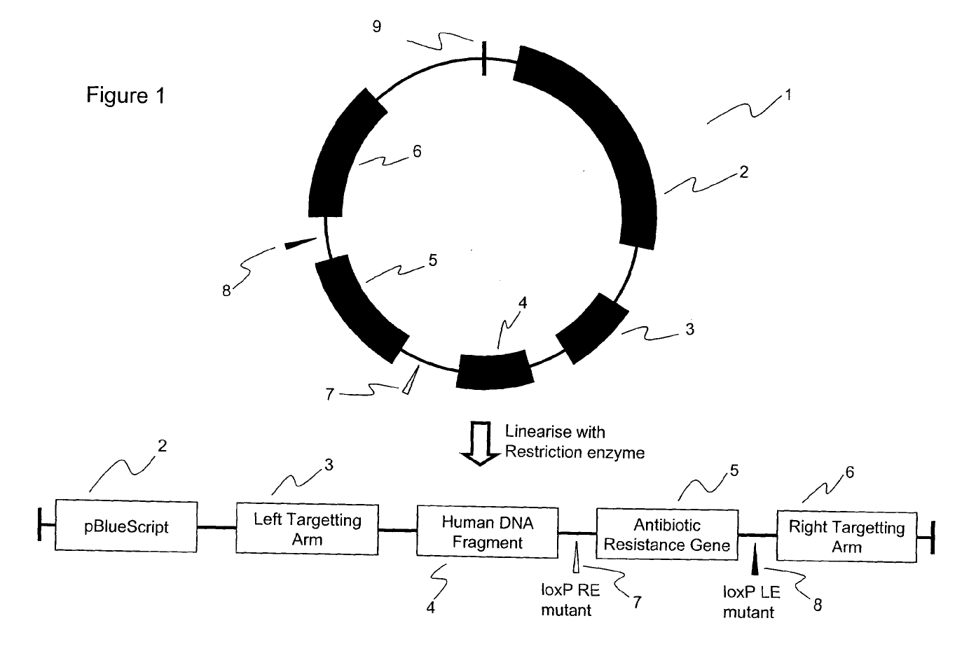

FIG. 1 shows, diagrammatically, a targeting vector that may be used to realise the current invention. The vector, generally 1, comprises the pBluescript sequence 2, of use in the bacterial stages of construction of the targeting plasmid, a left targeting arm 3, the human DNA fragment 4 to act as the reference material, an antibiotic resistance gene 5 and a right targeting arm 6. The targeting arms carry chicken DNA sequences for homologous recombination, enabling the integration of the human sequence and the antibiotic resistance gene into a specific site of the DT40 genome. The antibiotic resistance gene 5 may be flanked by mutant LoxP sites 7, 8. In the example shown in FIG. 1, there is a LoxP RE mutant 7 and a LoxP LE mutant 8. These LoxP sites enable the removal of the antibiotic resistance gene by use of the enzyme CRE Recombinase, once the vector sequences are integrated into the chicken genome. This technology is described in Arakawa et al, BMC Biotechnology 2001, 1:7. Situating the mutant LoxP sites flanking the antibiotics resistance gene enables the subsequent removal of the antibiotic resistance gene, facilitating the re-use of that antibiotic selection marker in any further gene-targeting events in the modified cell line. The targeting vector 1 also has a unique restriction enzyme site 9 to enable the vector to be linearised by cleavage with a restriction enzyme, prior to introducing it into the host DT40 cell lines by electroporation. Other methods of transfection will be apparent to the skilled addressee.

Typically each targeting arm 3, 6 would be 2-5 kilobases in size. In human DNA the human DNA fragment would typically be around 1 kilobase in size, and the variant base would be located towards the centre of the fragment.

In this example, the human genetic reference sequence 4 is to be inserted in a dispensable region of the DT40 genome. A suitable dispensable region is the genes coding for the high mobility group A (HMGA) family of non-histone chromosomal proteins, encoded by the two related genes, HMGA1 and HMGA2. It has been shown by Beitzel and Bushman (“Construction and analysis of cells lacking the HMGA gene family.” Nucleic Acids Res. 2003 Sep. 1:31(17):5025-32.) that the HMGA gene family is dispensable for growth in DT40 cells. They found no significant changes in the activity of approximately 4,000 chicken genes following deletion of either or both HMGA1 or HMGA2. They concluded that the HMGA proteins are not strictly required for growth control in DT40 cells. This region of the DT40 genome is thus a suitable target for insertion of the human genetic reference sequence 4. Others may readily be found by the skilled addressee, by reference to the literature (see e.g. Li Y, Strahler J R, Dodgson J B. “Neither HMG-14a nor HMG-17 gene function is required for growth of chicken DT40 cells or maintenance of DNaseI-hypersensitive sites.” Nucleic Acids Res. 1997 Jan. 15; 25(2):283-8) or sequence databases.

Thus, the left targeting arm 3 and right targeting arm 6 may be constructed by reference to the published sequence of the HMGA gene family. The plasmid 14 may therefore be constructed using the well-established pBluescript constructs using these targeting arms 3 and 6 and the human genetic reference sequence 4. A suitable antibiotic resistance marker 5 will be evident to the skilled addressee and would include, for example, Neomycin, Puramycin or Plasticidin. These antibiotic resistance genes may be driven by the chicken B-actin promoter. Detailed protocols for construction of the plasmid will be immediately evident to the skilled addressee given this teaching.

Following construction of the plasmid 1, the skilled addressee will be readily able to transform the host DT40 cells by, for example, linearisation of the plasmid 1 with restriction enzyme specific for the restriction site 9 and introduce the linearised construct into DT40 by e.g. electroporation.

### Embodiment 2

This embodiment demonstrates how the invention may be worked to create a di-allelic genetic reference standard.

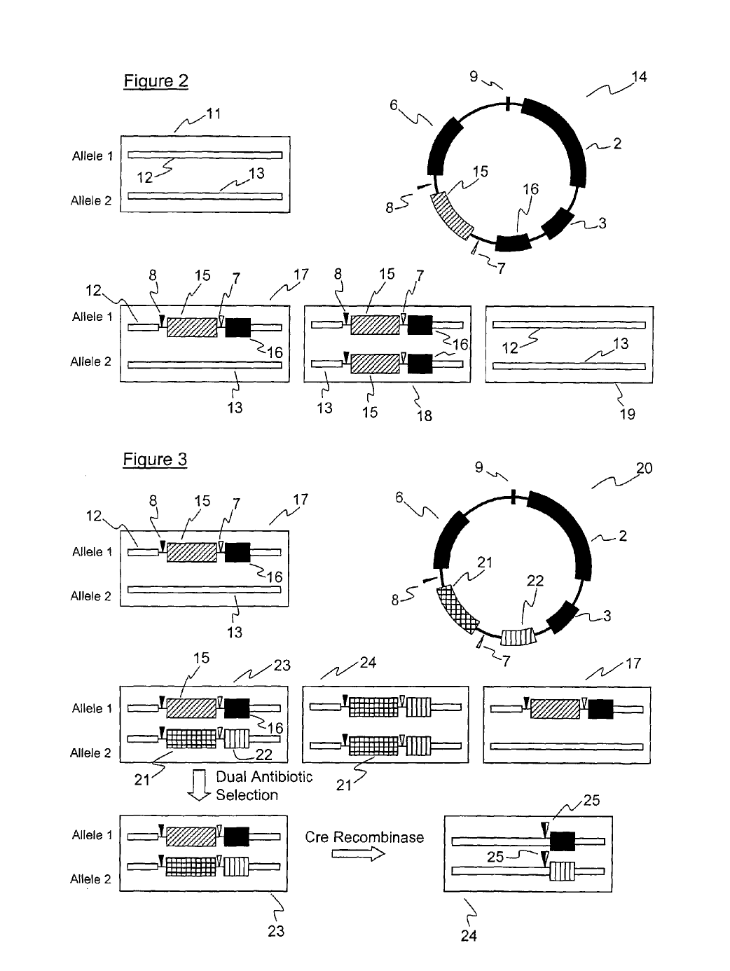

FIG. 2 illustrates the first part of the cell line construction process. There is illustrated a plasmid 14 containing the pBluescript sequences 2, a left targeting arm 3 and a right targeting arm 6, and an antibiotic resistance marker 15 with appropriate promoters. The plasmid also has the mutant LoxP sites 7 and 8. The first human genetic reference sequence which will make up one of the alleles is illustrated as 16.

Host DT40 cells to be transformed in this first step are represented by 11 with the two native chicken DNA alleles 12 and 13 illustrated at the cloning site.

Following recombination after introduction of the plasmid 14 into the host cells 11, three cell types may be present. Cell type 17 represents a hemizygote containing the integrated human genetic reference sequence 16 and the antibiotic resistance marker 15 flanked by the two mutant LoxP sites 7 and 8. There may also be cells 18 homozygotic for the human genetic reference sequence 16, the antibiotic resistance marker 15 and the two mutant LoxP sites 7 and 8. Finally, there will be a population of cells 19 that has not been transformed.

The untransformed cells 19 may be eliminated by selection with the appropriate antibiotic leaving a mixed population of hemizygotes 17 and homozygotes 18. There may also be some cells present (not illustrated) containing additional copies of plasmid-derived genetic material by random integration (i.e. not at the target site). Clonal sub populations from this mixed culture may be readily produced by the skilled addressee by, for example, the use of a dilution technique or flow cytometry assisted cell sorting. (If it is desired to use flow cytometry to produce a clonal sub-population, then the gene for green fluorescent protein—or a similar fluorescent protein—may be conveniently attached to one end of the targeting construct, so that it is retained, and expressed, in random integrants, but eliminated from targeted integrants, so allowing the separation of the desired cells by Fluorescence Assisted Cell Sorting). These clonal cell lines may then be screened using eg. PCR and Southern Blotting to choose the line 17 that is hemizygous for the human genetic reference sequence 16.

This hemizygous cell line 17 is then used as the host for the second stage of the procedure, which is illustrated in FIG. 3. A second plasmid 20 is used in this stage of the process. This again may contain the pBluescript elements 2, the left and right targeting arms 3 and 6, and the unique restriction site 9. This second plasmid 20 also contains a second antibiotic resistance marker 21, distinct from the first marker 15 illustrated in FIG. 2. This second marker 21 may again be flanked by the mutant LoxP sites 7 and 8. Included in this second plasmid 20 is the second human genetic reference sequence 22. This could be identical to that used in the first stage to create a homozygous cell line, or could be the human genetic reference sequence without the SNP to create a heterozygous standard.

Using the hemizygous cell line 17 as the starting host, recombination may be performed using this second plasmid 20 as before. Predominant cell types resulting from this will be heterozygotes 23 containing both antibiotic resistance markers 15, 21 and both human genetic reference sequences 16 and 22. There may also be some cell types that are homozygous 24 for the second marker 21 and sequence 22 where these sequences have replaced those inserted in the first stage. There will also be cells that are hemizygotic 17, i.e. where no recombination has occurred in this second stage. These will be selected against bu the presence of the antibiotic. The heterozygotic cells 23 may be selected by the use of both antibiotic selection markers. The resistance markers 15 and 21 may then be removed from these heterozygotic cells 23 by the use of Cre Recombinase to produce the genetic reference standard cells 24 containing just the two reference sequences and the non-mutant LoxP sites 25.

The invention is described in the claims that follow, in which the term “human genetic reference sequence” comprises a human DNA sequence containing at least one genetic variant whose presence in the DNA of a human subject is indicative of a pathological condition, a predisposition to a pathological condition, or a predisposition to an adverse reaction to external stimuli. The genetic variant may also be indicative of a patient's likely response to a therapeutic intervention, i.e. a variant used in pharmacogenomic analysis. The said genetic variant comprises a change, insertion or deletion of one or more bases with respect to the most common sequence in a human population, and includes single nucleotide polymorphisms (SNP), mutations, base or sequence insertions, base or sequence deletions and a change in tandem repeat length. In one embodiment of the invention, when the standard is used as a control, the “variant” may itself comprise the most common sequence. The reference sequence is characterised in that its total length is at least 35 bases, and does not exceed 30 kilobases. For some applications, the minimum length of the reference sequence may need to be more than 100 bases, to allow detection in an assay in which the reference is to be used. A length of 500 bases will be sufficient for almost all applications, but, within this teaching, the skilled addressee may readily determine an appropriate length by routine experiment, and without further inventive thought.

What is claimed is:

1. A genetic reference standard comprising at least one human genetic reference sequence cloned into a non-mammalian animal cell line.

2. The genetic reference standard of claim 1 wherein the animal cell line is an avian cell line.

3. The genetic reference standard of claim 2 wherein the cell line is a chicken (Gallus spp.) cell line.

4. The genetic reference standard of claim 1 wherein the cell line is a B-cell line.

5. The genetic reference material of claim 3 wherein the chicken cell line is the chicken DT40 cell line.

6. The genetic reference standard according to claim 1 wherein the at least one human genetic reference sequence is cloned into a dispensable region of the cell's genome.

7. The genetic reference standard according to claim 1 wherein the at least one human genetic reference sequence is cloned into a non-expressed region of the cell's genome.

8. The genetic reference standard according to claim 1 wherein the cloned cell line is diploid with respect to the human genetic reference sequence.

9. The genetic reference standard according to claim 1 wherein the at least one human genetic reference sequence is a plurality of human genetic reference sequences.

10. The genetic reference standard according to claim 1 wherein the at least one human genetic reference sequence is not a functional chromosome.

11. A method of detecting a genetic variant in a sample containing human DNA comprising:
performing a test, responsive to DNA sequence, on said sample;
performing the same test on a reference sample embodying the genetic variant to be detected;
comparing the test results obtained from said sample and said reference sample to determine the presence or absence of said genetic variant; wherein said reference sample is a genetic reference standard according to claim 1.
